(19) United States
(12) Patent Application Publication
(10) Pub. No.: US 2010/0257634 A1
(43) Pub. Date: Oct. 7, 2010

(54) BIOASSAY FOR GENE SILENCING CONSTRUCTS

(75) Inventors: ANA M. BAILEY, RALEIGH, NC (US); CHARLES NIBLETT, RALEIGH, NC (US)

Correspondence Address: THE FEDDE LAW FIRM, 218 ORCHARD AVE., WEBSTER GROVES, MO, 63119, US

(73) Assignee: VENGANZA INC., RALEIGH, NC (US)

(21) Appl. No.: 12/753,901

(22) Filed: Apr. 4, 2010

Related U.S. Application Data
(60) Provisional application No. 61/166,666, filed on Apr. 3, 2009.

(51) Int. Cl. A01H 1/00; A61K 49/00; C12Q 1/02; A61P 43/00

(52) U.S. Cl. 800/278; 424/9.2; 435/29

(57) ABSTRACT

The invention provides constructs and methods of screening for constructs useful in conferring resistance in plants to pests by gene silencing. The invention also provides pest-resistant plants transformed with the present constructs. One screening method of the invention comprises the steps of: selecting at least one pest target nucleotide sequence, producing a plurality of dsRNA test agents that target the pest target nucleotide sequence, testing and scoring the plurality of dsRNA test agents for toxicity to the pest, and producing a silencing construct based on a superior-scoring test agent.

This application claims the benefit of U.S. Provisional Application Ser. No. 61/166,666 filed 3 Apr. 2009, hereby incorporated by reference herein in its entirety.

### TECHNICAL FIELD

The present invention relates to the field of genetics. More specifically, the present invention relates to constructs useful in conferring resistance in plants to pests by gene silencing and methods for screening for useful constructs.

### BACKGROUND

Plants represent a major economical system for large-scale production of proteins and recombinant proteins that are important in pharmaceutical and industrial uses (Ma et al. 2003)

Commercial crops are often the targets of pest attack. Substantial progress has been made in the last few decades towards developing more efficient methods and compositions for controlling plant pests. Chemical pesticides have been effective in various pest infestations. However, there are several disadvantages to using chemical pesticidal agents. Applications of chemical pesticides are intended to control pests that are harmful to various crops and other plants. However, because of the lack of selectivity, the chemical pesticidal agents exert their effects on non-target flora and fauna as well, often effectively sterilizing a field for a period of time over which the pesticidal agents have been applied. Chemical pesticidal agents persist in the environment and generally are slow to be metabolized, if at all. They accumulate in the food chain, and particularly in the higher predator species. Accumulations of these chemical pesticidal agents results in the development of resistance to the agents and, in species higher up the evolutionary ladder, act as mutagens and/or carcinogens often causing irreversible and deleterious genetic modifications. Thus there has been a long felt need for environmentally friendly methods for controlling or eradicating insect infestation on or in plants, i.e., methods which are selective, environmentally inert, non-persistent, and biodegradable, and that fit well into pest resistance management schemes.

Compositions that include Bacillus thuringiensis (B.t.) bacteria have been commercially available and used as environmentally safe and acceptable insecticides for more than thirty years. The insecticidal effect of Bt bacteria arises as a result of proteins that are produced exclusively by these bacteria that do not persist in the environment, that are highly selective as to the target species affected, exert their effects only upon ingestion by a target pest, and have been shown to be harmless to plants and other non-targeted organisms, including humans. Transgenic plants containing one or more genes encoding insecticidal B.t. protein are also available in the art and are remarkably efficient in controlling insect pest infestation. A substantial result of the use of recombinant plants expressing Bt insecticidal proteins is a marked decrease in the amount of chemical pesticidal agents that are applied to the environment to control pest infestation in crop fields in areas in which such transgenic crops are used. The decrease in application of chemical pesticidal agents has resulted in cleaner soils and cleaner waters running off of the soils into the surrounding streams, rivers, ponds and lakes. In addition to these environmental benefits, there has been a noticeable increase in the numbers of beneficial insects in crop fields in which transgenic insect resistant crops are grown because of the decrease in the use of chemical insecticidal agents.

Double-stranded RNA (dsRNA) mediated inhibition of specific genes in various pests has been previously demonstrated. dsRNA mediated approaches to genetic control have been tested in the fruit fly Drosophila melanogaster (Tabara et al., (1998) Science 282:430-431). Tabara et. al. describe a method for delivery of dsRNA involved generating transgenic insects that express double stranded RNA molecules or injecting dsRNA solutions into the insect body or within the egg sac prior to or during embryonic development. Research investigators have previously demonstrated that double-stranded RNA mediated gene suppression can be achieved in nematodes either by feeding or by soaking the nematodes in solutions containing double stranded or small interfering RNA molecules and by injection of the dsRNA molecules. Rajagopal et. al. described failed attempts to suppress an endogenous gene in larvae of the insect pest Spodoptera litura by feeding or by soaking neonate larvae in solutions containing dsRNA specific for the target gene, but was successful in suppression after larvae were injected with dsRNA into the hemolymph of 5.sup.th instar larvae using a microapplicator (J. Biol. Chem., 2002, 277:46849-46851).

Similarly, Mesa et al. (US 2003/0150017A1) prophetically described a preferred locus for inhibition of the lepidopteran larvae Helicoverpa armigera using dsRNA delivered to the larvae by ingestion of a plant transformed to produce the dsRNA.

Niblett (WO2006047495) demonstrated that plants can be transformed with a construct that produces transcripts that form double-stranded RNA molecules with homologies to a pest essential gene. Through mechanisms only partially understood, a combination of plant and pest machinery contributes to the rendering the pest nonpathogenic by “knock-out” of an essential pest gene.

Raemaekers et al. (US 2009/030079 A1) describes methods of screening dsRNA complexes for toxicity to pests such as nematodes and beetles. However, Raemaekers et al. do not teach screening methods which replicate the production of RNAi agents such as dsRNA from a host plant.

There are many known and unknown factors that modulate the efficacy of a silencing construct. Accordingly, construct design and optimization can require expensive and long-term greenhouse or field trials. What is needed in the art is a flexible and rapid system for screening of constructs useful for producing pest resistant plants.

### SUMMARY OF THE INVENTION

A method has now been discovered that identifies constructs useful for conferring pest resistance in a plant.

The present invention provides a method comprising the steps of:
(a) selecting at least one target pest nucleotide sequence;
(b) producing a plurality of test agents, wherein each test agent comprises an antisense RNA molecule corresponding to the at least one target pest nucleotide sequence, wherein the test agent optionally further comprises a sense RNA molecule;
(c) testing each of the plurality of test agents, wherein the testing of each of the plurality of test agents comprises:
   i. administering the test agent to a pest;
   ii. measuring a toxic effect of the test agent on the pest; and wherein the measured toxic effect of a first test agent of the plurality is greater than the measured toxic effect of a second test agent of the plurality; and
(d) after the testing step, producing a silencing construct comprising an antisense sequence, wherein the silencing construct exhibits greater homology to the first test agent than to the second test agent, optionally wherein the silencing construct further comprises a sense sequence.

Optionally, the method further comprises the step of incubating the sense and antisense RNA molecules under conditions that allow formation of a double stranded molecule (“dsRNA”).

Optionally, the dsRNA molecules are digested into smaller dsRNA molecules before the administration step (e.g. by dicer).

Optionally, the sense and the antisense RNA molecules are administered with an RNA stabilizer.

Optionally, the sense and the antisense RNA molecule are administered with an RNA uptake enhancer.

Optionally, sequences predicted to be useful according to the present invention (e.g. a sequence homologous to the first test agent) are used in a gene silencing construct to transform a plant to confer pest resistance.

Optionally, the administration step mimics delivery of test agents when expressed by the plant. Optionally, administration does not comprise expressing the test agent in a plant. Optionally, the administration comprises feeding or incubating. Optionally, the test agents are digested with dicer (e.g. eukaryotic dicer).

Surprisingly, in embodiments of the present invention, there is a positive correlation between the toxic effect measured in step c and pest resistance in a plant transformed with the silencing construct.

### DETAILED DESCRIPTION OF THE INVENTION

As used here, the following abbreviations and definitions apply.

“Constructs useful for conferring pest resistance in a plant”, as used here, includes genes, gene fragments, intervening sequences, coding sequences, and the like.

Czm=Cercospora zeae-maydis

Fsg=Fusarium solani glycines

“Examplary” means a non-limiting example.

“Gene silencing”, as used here means any method of postranscriptionally reducing gene expression by a method that involves a polynucleotide that hybridizes to an mRNA or rRNA molecule and the subsequent hydrolysis of that RNA. Examples of such gene silencing include the technology of antisense, RNAi, siRNA, siNA, dsRNA, miRNA, short hairpin RNA, and ribozyme.

“Gene silencing construct” or “silencing construct”, as used here, means a construct useful for transforming a plant and that contains an element for gene silencing a plant pest gene.

Gg=Glomerella graminicola

Gm=Gibberella moniliformis

GUS=Escherichia coli β-glucuronidase

Gz=Gibberella zeae

Pcs=Puccinia sorghi

Pp=Phakopsora pachyrhizae

Ps=Phytophthora sojae

“RNA molecules”, as used here, is meant to embrace sense RNA molecules and antisense RNA molecules of the present invention irrespective of length (e.g. digested or not digested) and irrespective of single or double strandedness. RNA molecules as taught here correspond to the target nucleotide sequence. By “correspond” it is meant that an RNA molecule has sufficient homology such that hybridization to a target nucleotide sequence might reasonably be predicted.

Ss=Sclerotinia sclerotiorum

“siRNA” means short interfering RNA and is meant to embrace naturally produced siRNAs or synthetic siRNAs. Synthetic siRNAs can be produced by recombinant or chemical synthesis or by digestions of dsRNAs as taught herein.

“Target nucleotide sequence” is a sequence contained in a gene whose expression is to be selectively inhibited by gene silencing or by the screening methods taught herein. A target nucleotide sequence can also be a sequence in an unprocessed RNA molecule, an mRNA, or a ribosomal RNA sequences.

Xcc=Xanthomonas campestris pv campestris

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

Identifying Constructs Useful for Conferring Pest Resistance in a Plant

The methods of the present invention are useful for identifying known or unknown genes that, when incorporated into a silencing construct and transforming a plant therewith, confers to the plant, pest resistance. Moreover, the present invention provides for identifying regions within a gene that are especially useful for conferring resistance by gene silencing. For example, siRNAs (e.g. synthetic siRNAs) can be individually tested to probe for regions that are superior or inferior in gene silencing. Thus, constructs used to transform a plant to confer pest resistance can be designed to be absent sequences of the target that proved to be inferior in causing toxic effects in a pest by the present screening methods and/or can contain one or more copies of gene sequences of the target that proved to be superior in causing toxic effects by the present screening methods.

In one embodiment, the present invention provides for the identification of regions of a target gene or genes between about 10 and about 600 nucleotides in length, or between about 15 and 400 nucleotides in length, or between about 15 and any of about 300 or about 200 or about 100 or about 50 or about 25 nucleotides in length.

Target Nucleotide sequence selection

One or more target nucleotide sequences are selected according to the present invention. This selection step can be accomplished by the skilled, for example, by random selection or by consideration of the target pest, targetable genes, physicochemical properties of the regions of stability, and bioinformatic analyses. For example, an essential gene of the pest or a related pest can be selected as the target nucleotide.

The skilled artisan will now readily recognize that target nucleotide sequence selection can be made by well-understood methodologies in the art, e.g. Current Protocols in Bioinformatics (Published by John Wiley & Sons).

Potential targets for pest control can be identified in silico using a comparative genomics approach based on predicted functions and homology to genes from model organisms which are known to be essential for viability of the organism or crucial for important aspects of its pathogenicity (Lavorgna, G., Boncinelli, E. Wagner, A., and Werner, T. (1998). Detection of potential target genes in silico Trends in Genetics 14(9), 375-376). Such targets can then be validated by functional disruption using RNA interference or by studying knock out mutants of the target gene (WO 00/01846; Bosher, J. M. and Labouesse, M. (2000) RNA interference: genetic wand and genetic watchdog. Nature Cell Biology 2(2), E31-E36; Bird, D. M., Opperman, C. H., Jones, S. J. M., and Baillie, D. L. (1999) The Caenorhabditis elegans genome: A guide in the post genomics age. Annual Review of Phytopathology 37, 247-265).

Automated methods now exist to further aid in the target nucleotide sequence selection. For example, Yaun et al. (“siRNA Selection Server: an automated siRNA oligonucleotide prediction server” Nucleic Acids Research 2004 32 [Web Server Issue]:W130-W134) show that not all 21 nucleotide fragments of a target gene are equally effective and that superior fragments can be selected by aid of an algorithm and can be accessed at http://jura.wi.mit.edu/bioc/siRNA. Such algorithm is based upon several general rules such (1) a run of four or more Ts or As should be excluded under some circumstances because four or five Ts in a row is the transcription terminator signal for pol III; (2) if it is desired to design hairpin RNA expression vectors that are expressed from pol III promoters (U6, H1, or tRNA promoter), pol III terminator signals must be excluded from the sense or anti-sense strand; (3) four or more Gs in a row should be excluded because oligoG-containing RNAs may form tetraplexes and are difficult to chemically synthesize with some types of RNA chemistry; and (4) GC rich sequences form more stable duplexes than those that are AT rich, thus more than seven G/C pairs in a row would be suboptimal.

Producing a Sense RNA Molecule and an Antisense RNA Molecule

RNA molecules as taught here can be produced by any method known to the skilled artisan.

RNA molecules can be synthesized either in vivo or in vitro. Endogenous RNA polymerase of a cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands).

RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see WO 97/32016; U.S. Pat. Nos. 5,593,874, 5,698,425, 5,712,135, 5,789,214, and 5,804,693; and the references cited therein).

For RNA synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

Synthetic siRNAs of known sequence can be synthesized and tested individually or can be tested as a “pool” of siRNAs (e.g. as prepared by enzymatic cleavage of known dsRNAs).

The skilled artisan will now readily recognize that RNA molecules can be made by any well-understood methodologies in the art, e.g. as taught in Current Protocols in Molecular Biology (Published by John Wiley & Sons).

RNA Stabilizers

Optionally, the sense RNA molecules and the antisense RNA molecules are administered with an RNA stabilizer.

By way of example, an RNA stabilizer useful according to the present invention includes RNA chemical modification to increase stability. Examples of such stabilizing means are set forth in, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61).

Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). Additional modified nucleotides are as follows (this list contains forms that are modified on either the backbone or the nucleoside or both, and is not intended to be all-inclusive): 2′-O-Methyl-2-aminoadenosine; 2′-O-Methyl-5-methyluridine; 2′-O-Methyladenosine; 2′-O-Methylcytidine; 2′-O-Methylguanosine; 2′-O-Methyluridine; 2-Amino-2′-deoxyadenosine; 2-Aminoadenosine; 2-Aminopurine-2′-deoxyriboside; 4-Thiothymidine; 4-Thiouridine; 5-Methyl-2′-deoxycytidine; 5-Methylcytidine; 5-Methyluridine; 5-Propynyl-2′-deoxycytidine; 5-Propynyl-2′-deoxyuridine; N1-Methyladenosine; N1-Methylguanosine; N2-Methyl-2′-deoxyguanosine; N6-Methyl-2′-deoxyadenosine; N6-Methyladenosine; O6-Methyl-2′-deoxyguanosine; and 06-Methylguanosine. A variety of chemical synthetic approaches are available for the conjugation of additional moieties to nucleic acids. For example, one may synthesize nucleic acid-lipid, nucleic acid-sugar conjugates (see, e.g., Anno et al. Nucleosides Nucleotides Nucleic Acids. May-August 2003; 22(5-8):1451-3; Watal et al. Nucleic Acids Symp Ser. 2000; (44):179-80), nucleic acid-sterol conjugates or conjugates of other relatively fat soluble hydrophobic moieties such as vitamin E, dodecanol, arachidonic acid, folic acid and retinoic acid (see, e.g., Spiller et al., Blood. Jun. 15, 1998; 91(12):4738-46; Bioconjug Chem. March-April 1998; 9(2):283-91; Lorenz et al. Bioorg Med Chem. Lett. Oct. 4, 2004; 14(19):4975-7; Soutschek et al. Nature. Nov. 11, 2004; 432 (7014):173-8). See also the review of nucleic acid conjugates in Manoharan Antisense Nucleic Acid Drug Dev. April 2002; 12(2):103-28.

In order to further enhance the stability of the dsRNA molecules, the optional 3′ overhangs can optionally be stabilized against degradation. In one embodiment, the RNA molecules are stabilized by including purine nucleotides, such as adenosine or guanosine nucleotides. Alternatively, substitution of pyrimidine nucleotides by modified analogues (e.g., substitution of uridine nucleotide 3′ overhangs by 2′-deoxythyinidine) is tolerated and does not affect the efficiency of RNAi. The absence of a 2′ hydroxyl significantly enhances the nuclease resistance of the overhangs.

Other RNA stabilizers include chemical modifications described by WO 2004/029212.

By way of example, an RNA stabilizer useful according to the present invention includes liposomes to encapsulate the RNA molecules. Examples of such useful liposomes are described by US2005002998.

Also by way of example, an RNA stabilizer useful according to the present invention includes various chemistries and non-canonical base pairs (e.g. mismatches and/or wobble base pairs) described by US2006217331.

As another example, an RNA stabilizer can be non-canonical base pairing (e.g. U-A, U-G, U-C, U-U) further comprising chemical modifications (e.g. 2′ substituent s or replacing the ribose with a hexose sugar) as described in WO2005115481.

As another example, an RNA stabilizer can be lessening base pair strength between 5′- or 3′-terminal of an RNA in comparison to 3′- and 5′-terminal of RNA strand.

RNA Uptake Enhancer.

Optionally, the RNA molecules are administered with an RNA uptake enhancer. RNA uptake enhancers include RNA conjugates. Conjugates can be selected based on the ability of the molecules to be selectively transported into specific cells, for example via receptor-mediated endocytosis. By attaching RNA molecules to molecules that are actively transported across the cellular membranes, the effective transfer of RNA molecules into cells or specific cellular organelles can be realized.

Optional RNA uptake enhancers include molecules that are able to penetrate cellular membranes without active transport mechanisms, for example, various lipophilic molecules, can be used to deliver RNA molecules of the present invention. Examples of molecules that can be utilized as conjugates include but are not limited to peptides, hormones, fatty acids, vitamins, flavonoids, sugars, reporter molecules, reporter enzymes, chelators, porphyrins, intercalcators, and other molecules that are capable of penetrating cellular membranes, either by active transport or passive transport.

Optional RNA uptake enhancers include molecules that modulate permeability of the epithelial cell barrier complex. By way of example, such enhancers can be polysaccharides such as glycosaminoglycans and agents that modify cell surface glycosaminoglycans such as glycosaminoglycan-degrading enzymes to modulate intercellular junctions. Examples of these types of RNA uptake enhancers are described in WO2006088491.

Optional RNA uptake enhancers include polycationic polymers. For example, Ryser et al., International PCT Publication No. WO 79/00515 describes the use of high molecular weight lysine polymers for increasing the transport of various molecules across cellular membranes. Rothbard et al., International PCT Publication No. WO 98/52614, describes certain methods and compositions for transporting macromolecules across biological membranes in which the macromolecule is covalently attached to a transport polymer consisting of from 6 to 25 subunits, at least 50% of which contain a guanidino or amidino side chain. The transport polymers can be polyarginine peptides composed of all D-, all L- or mixtures of D- and L-arginine. Rothbard et al., U.S. Patent Application Publication No. 20030082356, describes certain poly-lysine and poly-arginine compounds for the delivery of drugs and other agents across epithelial tissues, including the skin, gastrointestinal tract, pulmonary epithelium and blood brain barrier. Wendel et al., U.S. Patent Application Publication No. 20030032593, describes certain polyarginine compounds. Rothbard et al., U.S. Patent Application Publication No. 20030022831, describes certain poly-lysine and poly-arginine compounds for intra-ocular delivery of drugs. Kosak, U.S. Patent Application Publication No. 20010034333, describes certain cyclodextran polymers compositions that include a cross-linked cationic polymer component. Lewis et al., U.S. Patent Application Publication No. 20030125281, describes certain compositions consisting of the combination of siRNA, certain amphipathic compounds, and certain polycations.

Other useful RNA uptake enhancers of the polycationic polymers type are described by US2005222064.

Other useful RNA uptake enhancers include peptides conjugated to RNA molecules as described by US2004204377.

The skilled artisan will now recognize that certain RNA uptake enhancers can also be (i.e. function as) RNA stabilizers and certain RNA stabilizers can also be RNA uptake enhancers. Generally an RNA stabilizer slows the decrease in ability of RNA molecules to cause toxic effects when administered to a pest. Generally an RNA uptake enhancer results in RNA molecules having a given level of toxicity at a reduced concentration.

Formation of Double Stranded Molecules

Sense RNA molecules and antisense RNA molecules according to the present invention optionally form double stranded regions. The double-stranded structures may be formed by a single self-complementary nucleic acid strand or two, noncontiguous complementary nucleic acid strands

Double stranded region means a region of a polynucleotide wherein the nucleotides are capable of hydrogen bonding to each other. Such hydrogen bonding can be intramolecular or intermolecular (e.g. single transcription unit forming a double stranded region with the so-called hairpin or two transcription units that align appropriately for complementary sequences to hydrogen bond). To be a double stranded region, according to the present invention, it is not necessary for 100% of the nucleotides to be complementary and hydrogen bonded within a region. It is merely necessary for sufficient base pairing to occur to give the RNA a substantial double stranded character (e.g. an indicative melting point).

In certain embodiments, at least one strand of the double stranded RNA molecules has a 3′ overhang from about 1 to about 6 nucleotides in length, or from 2 to 4 nucleotides in length. In certain embodiments, one strand has a 3′ overhang and the other strand is blunt-ended or also has an overhang. The length of the overhangs may be the same or different for each strand.

It is well known that various conditions may be employed to achieve sufficient formation of dsRNA molecules. Factors such as the length and nature of the RNA and the concentration of the salts and other components (e.g., the presence or absence of formamide, dextran sulfate, polyethylene glycol) are considered and the hybridization solution may be varied to generate the desired stringency of hybridization. In addition, the art provides conditions that promote hybridization (e.g., temperature of the hybridization and/or wash steps, the use of formamide in the hybridization solution, etc.).

Digesting dsRNA

RNA molecules can be cleaved by enzymatic digestion to shorter molecules (e.g. siRNA). Such digestion can be performed on single stranded RNA molecules or RNA molecules with double stranded regions.

Double stranded RNA molecules (i.e. dsRNA) can be digested to form double-stranded RNA fragments (e.g. siRNA), for example, by using a member of the Ribonuclease III (RNase III) family of double-stranded RNA-specific endoribonucleases.

Useful RNase III members are dicers. Generally, digestion with dicer produces dsRNA fragments of approx. 24 nt in length (approx. 20-basepair RNA duplex with a 2-nt 3′ overhang on each end).

Dicer can be any protein or polypeptide that cleaves long double stranded RNA, optionally using two distinct RNase domains (RNase IIIa and RNase IIIb; Zhang et al. (2004) Single Processing Center Models for Human Dicer and Bacterial RNase III, Cell 118: 57-68).

Dicer can be any polypeptide that has the Dicer activity of a Dicer protein, e.g. it can be a partial polypeptide fragment that has at least the Dicer activity of a Dicer protein, or a protein comprising such polypeptides (for example, a full-length Dicer protein). The term “Dicer activity” typically refers to activities of digesting a long double-stranded RNA into double-stranded RNA fragments of 21-25 nucleotides. In general, Dicer activity can be examined by measuring RNaseIII activities. The activity can be assessed or measured by a method known to those skilled in the art, for example, an in vitro processing assay. For example, the in vitro processing assay can be carried out by the procedure described below in Examples.

There is no limitation as to the origin of Dicer protein to be used in the present invention. For example, human Dicer protein can be suitably used.

Dicer of the present invention can be any recombinant Dicer protein; e.g., Myers et al., 2003, Nature Biotechnol 21, 324-8; Beach et al., 2003, US Pat Appl Publ US2003/0084471; Zhang et al., 2002, EMBO J. 21, 5875-85; Dicer siRNA generation kit (Gene Therapy Systems, Inc., San Diego, Calif., Catalog No.T51001).

Dicer can be used with additional proteins that modulate Dicer activity, e.g. the R2D2 protein described by Wang et al. (US 20050069990) that forms a complex comprising the R2D2 protein and the Dicer protein. The Dicer protein and the R2D2 protein may be coexpressed in insect cells, such as S2, Sf9 or Hi5 cells, using a baculovirus expression system.

Another useful RNase III member is bacterial RNase III which generally produces dsRNA fragments of about 13 nt in length (approx. 9-basepair duplex with a 2-nt 3′ overhang on each end).

The Dicer method of preparing siRNAs can be performed using a Dicer siRNA Generation Kit available from Gene Therapy Systems (San Diego, Calif.).

RNA Molecule Administration

The invention encompasses any suitable means to administer RNA molecules systemically and/or locally to a target site in a pest. Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct.

Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

RNA molecules may be incorporated into, or overlaid on the top of, the pest's diet. In another embodiment, the RNA may be sprayed onto a plant surface. RNA molecules can be expressed by microorganisms and the microorganisms can be applied onto a plant surface or introduced into a root or stem by a physical means such as an injection whereupon the pest is introduced to the plant (this, administering RNA molecules to the pest).

RNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. For example, in C. elegans, double-stranded RNA introduced outside the cell inhibits gene expression. Vascular or extravascular circulation, the blood or lymph system, the phloem, the roots, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Insects. RNA molecules of the present invention can be directly introduced into the cells of an insect, or introduced into an extracellular cavity, interstitial space, lymph system, digestive system, into the circulation of the insect through oral ingestion or other means that one skilled in the art may employ.

Administration of RNA molecules to adult aphids may comprise injecting (Mutti et al. “RNAi knockdown of a salivary transcript leading to lethality in the pea aphid, Acyrthosiphon pisum” Journal of Insect Science October 2006 Vol. 6, No. 38 pp; also see US 2006/0272049 A1 to Waterhouse et al.) or feeding (Waterhouse et al).

Administration of RNA molecules to aphid larvae may comprise injecting (Jaubert-Possamai et al. “Gene knockdown by RNAi in the pea aphid Acyrthosiphon pisum” BMC Biotechnology 2007, Vol. 7, No. 63) or feeding (WO 2007/080127 A2 to Raemaekers et al.)

Administration of RNA molecules to insect larvae may comprise injecting (Niimi et al. “Larval RNAi Applied to to the Analysis of Postembryonic Development in the Ladybird Beatle, Harmonia axyridis” Journal of Insect Biotechnology and Sericology 2005 Vol 74 Pages 95-102; also see Rajagopal et al. “Silencing of Midgut Aminopeptidase N of Spodoptera litura by Double-stranded RNA Establishes Its Role as Bacillus thuringiensis Toxin Receptor” The Journal of Biological Chemistry Vol. 277, No. 49, pp 46849-46851), feeding (Turner et al “RNA interference in the light brown apple moth, Epiphyas postvittana (Walker) induced by double-stranded RNA feeding” Insect Molecular Biology 2006 Vol. 15, No. 3, pp 383-391; also see US 2005/0095199 A1 to Whyward et al.; also see WO 2007/035650 A2 to Baum et al.; Raemaekers et al), or topically administering or soaking (Whyward et al.).

Administration of RNA molecules to adult insects may comprise injecting (Fuente et al “RNA interference for the study and genetic manipulation of ticks” TRENDS in Parasitology Vol. 23 No. 9 pp 427-433; also see Dong et al.), feeding (Fuente et al), or topically administering or soaking (Pridgeon et al. “Topically Applied AaelAP1 Double-Stranded RNA Kills Female Adults of Aedes aegypti” Journal of Medical Entomology 2008 Vol. 45, No. 3, pp 414-420).

Fungi. Administration of RNA molecules to fungi may comprise feeding or transformation (e.g. biolistics as taught by WO 2006/047495 A2 to Niblett) and methods taught herein. Administration can also be accomplished by methods described in Medical Mycology 45:211-220 (2007).

Nematodes. Administration of RNA molecules to larval or adult nematodes may comprise feeding, soaking, or injection (Montgomery “The Use of Double-Stranded RNA to Knock Down Specific Gene Activity” Methods in Molecular Biology, Vol. 260, pp 129-144).

Bacteria Administration of RNA molecules can be accomplished by the methods generally taught or specific disclosed by way of examples.

Plants. RNA molecules can be administered to plant pests of plants by biolistic administration to the apical meristem as taught by WO 2006/047495 to Niblett and elsewhere.

Measuring Toxic Effects in Pests

Toxic effects in a pest resulting from administering the RNA molecules of the present invention can be estimated or quantified by methods known to the skilled artisan. Examplary toxic effects are distortion of normal growth, growth habit, or morphology, death of the pest, reduced propagation, reduced pathogenicity.

Moreover, in some embodiments, toxic effect are measured in a portion of the pest (e.g. pest ex vivo) or in cultured pest cells or pest organs (e.g. in vitro). Accordingly, in this context, the term “pest” is meant to embrace these pest embodiments.

Fungi. Fungi can be cultured in various culture media and the toxic effects of RNA molecules can be determined following administration. Examplary toxic effects in fungi can include cell death, reduced germination, growth, sporulation, cell division, cell number, and pathogenicity and malformation of hyphae.

Other useful toxic effects include those described in Medical Mycology 45:211-220 (2007).

Fungi can be cultured on inoculated soybean leaves inserted in Magenta boxes containing water agar and the toxic effects of RNA molecules can be quantified, for example, as set forth below in the “Detached Soybean Leaf Assay”.

Nematodes. Toxic effects of RNA molecules on nematodes are well known and have been extensively described elsewhere.

Bacteria. Useful toxic effects of RNA molecules on bacteria include cell death or reduced colony formation as described elsewhere and below.

Screening and administration technologies are also disclosed by Raemaekers et al. (US 2009/030079 A1).

Predicting Utility of the Target Nucleotide Sequence

Measurements of toxic effects can be used to predict (or “score”) the utility of test agent in conferring resistance by way of gene silencing in a plant. For example, where toxic effects of administering RNA molecules to a pest are high, such sequences are predicted likely to be useful in a gene silencing construct. In general, the higher the measurement of toxic effects, the higher likelihood of usefulness and/or the greater the predicted level of efficacy when used in a gene silencing construct.

One skilled in the art will now readily appreciate that an algorithm can be developed to correlate the toxic effects observed according to this present invention with predicted efficacy of the RNA molecules in conferring pest resistance. Moreover, this algorithm can be modified as gene-silencing data is generated.

Pests

According to the present invention, constructs are evaluated for usefulness (or predicted usefulness) to confer pest resistance. Such a pest of the present invention can be any plant pest. By way of example, the plant pest can be an insect, nematode, bacterium, fungus, or other plant.

Examplary nematodes include soybean cyst nematode (Heterodera glycines), the Root knot nematode (Meloidogyne incognita) and the Golden nematode (Globodera rostochiensis), etc.

Examplary fungi, for the purpose of this application, are oomycetes that cause diseases such as late blight of potato (Phytophthora infestans), sudden oak death (Phytophthora ramorum) and damping off of seedlings (Pythium debaryanum)

Examplary pests that are “true fungi” are those that cause diseases such as head blight of wheat (Gibberella zeae), soybean sudden death syndrome (Fusaium solani glycines), anthracnose of corn (Glomerella graminicola) and soybean rust (Phakopsora pachyrhizi)

Examplary bacteria are those causing diseases such as citrus huanglongbing (Candidatus Liberibacter asiaticus), citrus canker (Xanthomonas axonopodis) and black rot of crucifers (Xanthomonas campestris)

Examplary pests that are Lepidoptera, are fall armyworm (Spodoptera frugiperda), the corn earworm (Helicoverpa zea) and lesser cornstalk borer (Elasmopalpus lignosellus)

Examplary pests that are Coleoptera are the corn root worm (Diabrotica virgifera virgifera).

Examplary pests include Colorado potato beetle (Leptinotarsa decemlineata).

Examplary pests that are Blattaria include cockroaches such as the American cockroach (Periplaneta americana) and the German cockroach (Blattella germanica).

Examplary aphids are green peach aphid (Myzus persicae) and melon aphid (Aphis gossypii).

Examplary pests that are Leafhoppers are the glassy-winged sharpshooter (Homalodisca vitripennis) and the beet leafhopper (Circulifer tenellis).

Examplary whiteflies are the sweetpotato whitefly (Bemisia tabaci) and the silverleaf whitefly (B. argentifolii).

Pest Resistance by Gene Silencing in a Plant

RNA sequences predicted to be useful in conferring pest resistance can be used to transform plants. Any suitable gene silencing method can be used including technology of antisense, RNAi, siRNA, siNA, dsRNA, miRNA, short hairpin RNA, and ribozyme.

Test agents and silencing constructs may include, for example, antisense RNA, dsRNA, siRNA, miRNA, shRNA, and other polynucleotide sequence containing a segment complementary to a target sequence, and capable of inhibiting or reducing expression of the target gene.

Plants can be made resistant to bacteria as described by Ream in WO0026346 by transformation with constructs optimized according to the present invention.

Plants can be made resistant to nematodes as described in US20050091713 by transformation with constructs optimized according to the present invention.

Plants can be made resistant to nematodes as described in EP1484415 by transformation with constructs optimized according to the present invention.

Plants can be made resistant to nematodes as described by Mesa in US 20030150017 by transformation with constructs optimized according to the present invention.

Plants can be made resistant to fungi as described in WO2005071091 by transformation with constructs optimized according to the present invention.

Plants can be made resistant to arthropods as described in US WO2003004644 by transformation with constructs optimized according to the present invention.

Plants can be made resistant to arachnids, insects, nematodes, protozoans, bacteria, and fungi as described by Fire in U.S. Pat. No. 6,506,559 by transformation with constructs optimized according to the present invention.

Plants can be made resistant to fungi, nematodes, bacteria, arthropods, insects, and combinations thereof as described by Niblett in US 20060095987 by transformation with constructs optimized according to the present invention.

Example Methods

In one embodiment the invention provides an example claim (EC) set forth below.

Example Claims
1. A method of identifying a construct useful for conferring pest resistance in a plant comprising the steps of:
   (a) selecting at least one target pest nucleotide sequence;
   (b) producing a plurality of test agents, wherein each test agent comprises an antisense RNA molecule corresponding to the at least one target pest nucleotide sequence, wherein the test agent optionally further comprises a sense RNA molecule;
   (c) testing each of the plurality of test agents, wherein the testing of each of the plurality of test agents comprises:
      i. administering the test agent to a pest;
      ii. measuring a toxic effect of the test agent on the pest;
   wherein the measured toxic effect of a first test agent of the plurality is greater than the measured toxic effect of a second test agent of the plurality; and
   (d) after the testing step, producing a silencing construct comprising an antisense sequence and optionally a sense sequence, wherein the silencing construct exhibits greater homology to the first test agent than to the second test agent.
2. The method of EC 1, further comprising a step of incubating the antisense RNA molecule and optional sense RNA molecule under conditions to allow formation of a double stranded complex before the administration step (c).
3. The method of EC 2 further comprising a step of digesting the double stranded molecule to form smaller double stranded RNA molecules before the administration step (c), optionally, wherein the digestion is accomplished by a dicer enzyme.
4. The method of EC 3, wherein the measured toxic effect of the first test agent is greater than that of the first test agent if not digested before the administration of step (c).
5. The method of EC 1 wherein the administration step (c) further comprises addition of an RNA stabilizer.
6. The method of EC 1 wherein the administration step (c) further comprises addition of an RNA uptake enhancer.
7. The method of EC 1, wherein the first test agent and the second test agent target the same gene.
8. The method of EC 7, wherein the first test agent and the second test agent correspond to overlapping segments of the same gene.
9. The method of EC 1, wherein the first test agent and the second test agent target different genes.
10. The method of EC 1, wherein the plurality of test agents comprises a number of test agents selected from the group consisting of: at least 10, at least 20, at least 50, and at least 96.
11. The method of EC 10, wherein, in addition to the first test agent, test agents in the top 5 percentile for toxicity are selected for silencing constructs.
12. The method of any of the above ECs, further comprising transforming a plant with the silencing construct.
13. The method of any of EC 12, wherein a plurality of plants are transformed with the silencing construct, optionally, wherein no plants are transformed with a second silencing construct corresponding to the second test agent, optionally, wherein less plants are transformed with a second silencing construct corresponding to the second test agent.
14. The method of EC 1, wherein the administration does not comprise expressing the test agent in a plant.
15. The method of any of ECs 1-14 wherein the pest is an insect, nematode, bacterium, fungus, or plant.
16. The method of any of ECs 1-14 wherein the pest is a fungus.
17. The method of any of ECs 1-14 wherein the plant is a corn plant, a soybean, a potato, a tomato, a banana, or a cotton plant.
18. The method of any of ECs 1-14 wherein the plant is a corn plant and the pest is a Fusarium, a Gibberella, a Cercospora, a Puccinia, a Bipolaris, or a Cochliobolus.
19. The method of any of ECs 1-14 wherein the plant is a corn plant and the pest is a Fusarium moniliforme, Gibberella zeae, a Cercospora zeae-maydis, a Puccinia sorghi, a Puccinia polysora, a Bipolaris maydis, or a Cochliobolus carbonum.
20. The method of any of ECs 1-14 wherein the plant is a soy bean and the pest is a Phytophthora, a Phakopsora, a Sclerotinia, or a Fusarium.
21. The method of any of ECs 1-14 wherein the plant is a soy bean plant and the pest is a Phytophthora sojae, a Phakopsora pachyrhiz, a Sclerotinia sclerotiorum, or a Fusarium solani f. sp. glycines.
22. The method of any of ECs 1-14 wherein the plant is a potato plant and the pest is a Phytophthora infestans, a Alternaria solani, or a Rhizoctonia solani.
23. The method of any of ECs 1-14 wherein the plant is a tomato plant and the pest is a Alternaria alternata f.sp. lycopersici, a Fusarium oxysporum f.sp. lycopersici, a Sclerotinia sclerotiorum, a Phytophthora infestans, or a Alternaria solani
24. The method of any of ECs 1-14 wherein the plant is a banana plant and the pest is a Fusarium, a Mycosphaerella, or a Colletotrichum.
25. The method of any of ECs 1-14 wherein the plant is a banana plant and the pest is a Fusarium oxysporum f. sp. cubense, a Mycosphaerella fijiensis, or a Colletotrichum musae
26. The method of any of ECs 1-14 wherein the plant is a cotton plant and the pest is a F. oxysporium f. sp. Vasinfectum, a Rhizoctonia solani, a Verticillium dahliae, a Ascochyta gossypi, or a Phymatotrichum omnivorum
27. The method of any of ECs 1-14 wherein the sense RNA molecule and antisense RNA molecule are joined through a phosphodiester linkage.
28. The method of any of ECs 1-14 wherein the administration step (c) is accomplished by feeding, injecting, bombardment, electroporation, or incubation.
29. The method of any of ECs 1-14 wherein the administration step (c) is accomplished by feeding or incubating.
30. The method of EC 29, wherein the pest is a fungus.

### EXAMPLES

### Example 1

### Testing Constructs against Phytophthora

Pests were cultured and tested with the gene constructs as set forth in Table 1.

TABLE 1
Phytophthora Species and Test Constructs.

| Pest | Test and culture methods | SEQ IDs tested |
|---|---|---|
| Phytophthora | Biolistics and imbibition; | 1 |
| nicotianae (Pn) | cultured on V8 media for | |
| | mycelia | |
| Phytophthora | Biolistics and imbibition; | 1, 2, 3, 5, 6, 8, 9, |
| sojae (Ps) | cultured on V8 media for | 10, 12, 14, 15, 16 |
| | mycelia | |
| Phytophthora | Biolistics and imbibition; | 1, 5, 6 |
| infestans (Pi) | cultured on V8 media for | |
| | mycelia | |
| Phytophthora | Imbibition; cultured on V8 | 6, 17, 18, 19 |
| cinnamomi | media for mycelia production | |

Preparation. For each fungus (plant pathogen), culture conditions (media composition, temperature, light) were specifically standardized to generate high number of spores or mycelium for toxicity testing with the RNA molecules.

Each selected target nucleotide sequence is essential to the fungus for development, reproduction or pathogenesis.

The selected target nucleotide sequences were amplified by PCR using specific oligonucleotides containing the T7 promoter sequence. The amplified DNA was purified and 1 microgram was subjected to transcription using the Ambion MEGAscript® High Yield Transcription Kit to produce dsRNA corresponding to the selected target nucleotide sequences.

For some of the examples, 30 micrograms of the dsRNA were subjected to digestion with RNase III using the Silencer® siRNA Cocktail Kit (RNase III) to produce a mixture of dsRNA fragments (siRNAs).

Administration by bombardment. For administration of RNA molecules by bombardment, dsRNA or siRNAs were coated onto gold particles. Bombardment was performed at 1000 psi into mycelia cultivated on agar plates and the fungi were grown for 2-3 days. Next, several 5 mm agar plugs from the bombarded area (3 cm) were transferred to agar plates and incubated for 2 days. The toxic effects of the RNA molecules were quantified by assessing radial growth and mycelia deformation or lysis.

Administration by imbibition. Alternatively, the dsRNA (or siRNA fragments thereof) were administered into spores (approximately 100,000 in number) or mycelia (5 mm agar plug) of the fungi by imbibition. Concentrations ranging from 10 to 50 micrograms of dsRNA and or 3-5 micrograms siRNA were tested to determine optimal concentration and toxic effects on the pathogen. Toxic effects were typically observed with 30 micrograms of dsRNA or 5 micrograms of the dsRNA fragments (which corresponded to the target genes).

After administration of the RNA molecules, the fungi were incubated for 3 to 24 hours. Next, spores or mycelia were harvested and plated onto specific media for 2-3 days at which time the number of colonies were determined. Alternatively, radial growth of mycelia was measured after 5-6 days incubation.

Results with Phytophthora spp. Treatment of P. nicotianae, P. sojae and P. infestans with SEQ ID 1 by either biolistics or imbibition did not cause observable differences in colony growth rate or hyphal morphology, but it did render all three species non-pathogenic on their usual host plants.

Treatment of P. nicotianae, P. sojae and P. infestans with SEQ IDs 5 and 6 by either biolistics or imbibition caused obvious and detrimental effects on these species. Biolistic transformation with constructs containing SEQ IDs 5 and 6 caused resulting colonies to be malformed, grow extremely slowly or die. Sporulation could not be induced from these transgenic cultures. Imbibition of SEQ IDs 5 and 6 by these species caused slow growth of the resulting colonies and severe malformation and distortion of hyphae viewed in the microscope.

Imbibition by P. sojae of SEQ IDs 8, 9, 10, 12, 14, 15, 16 had no detectable effect on colony growth rate or hyphal morphology.

Imbibition of SEQ IDs 17, 6, 18, 19 by P. cinnamomi, a fungal pest of avocado, caused 20%, 40%, 80% and nearly 100% reduction in colony growth, respectively. SEQ IDs 6, 18 and 19 caused severe malformation and distortion of hyphae, whereas it was barely evident with SEQ ID 17,

### Example 2

### Assay of Fungal Pests of Corn

The various dsRNA molecules were tested against the corn fungal pathogens indicated Table 2.

The dsRNA molecules were administered to the fungi by imbibition and toxicity tested by reduction in colony number as described in the text pertaining to Table 1.

The results in Table 2 are shown as the average of 3 replicates of 50 μl of solution containing 30 μg of dsRNAs

TABLE 2
Summary of Toxicity Tests of Various
dsRNAs on Corn Fungal Pathogens.

Corn Pathogens

| | | | | | |
|---|---|---|---|---|---|
| SEQ | RNA | Gz | Gm | Gg | Czm |

| | | |
|---|---|---|
| ID | source | % reduction in colony formation |

| | | | | | |
|---|---|---|---|---|---|
| 2 | GUS | 13^{X} | 13 | 3 | 13 |
| 4 | Pcs | 37 | 38 | 34 | 40 |
| 8 | Gz | 60 | 49 | 10 | 17 |
| 10 | Gz | 63 | 57 | 50 | 55 |
| 9 | Gz | 68 | ^{Y} | 49 | 45 |
| 11 | Gm | 55 | 59 | 53 | 50 |
| 14 | Cg | 50 | 50 | 58 | 45 |
| 13 | Czm | 60 | 55 | 45 | 58 |

### Example 3

### Detached Soybean Leaf Assay

A “Detached Soybean Leaf Assay” was developed to determine the effects of RNA molecules administered to Phakopsora pachyrhizi on the fungus' ability to infect soybeans. The toxic effects, as measured in this assay, relate to the reduced pathogenicity of the fungus. This assay was generally performed as follows:
a. Maintain Phakopsora pachyrhizi cultures on soybean leaves.
b. Wash spores from infected leaf with 0.01% Tween 20 in sterile water.
c. Concentrate spores in microfuge tube by centrifugation 2-3 min. at 10,000 rpm.
d. Dilute spores to 1 million/ml in 0.01% Tween 20 using a hemacytometer.
e. For each treatment incubate 10,000 spores overnight in 100 μl of Tween 20 containing either 30 μg dsRNA, 5 μg of siRNA or no RNA.
f. Transfer treated spores to the midrib of a water-misted soybean leaf.
g. Cover with a second leaf to make a “sandwich” and disperse the spores.
h. Incubate in a water-misted and sealed plastic bag at 25C for 48 hours in the dark.
i. Transfer leaves to a Magenta Box containing 50 ml of water agar by inserting petioles into the agar and incubate in light at 25C.
j. Pustules appear in 8-10 days.
k. Compare pustule numbers with controls containing unrelated RNA sequences or no RNA.

### Example 4

### Detached Soybean Leaf assay—Example II

The detached soybean leaf assay is described in detail below.

Cultivation of fungus. Phakopsora pachyrhizi cultures were grown in the laboratory on detached soybean leaves maintained in sealed Magenta boxes or Mason jars containing 50 ml of water agar. Leaves from greenhouse-grown plants were misted with deionized water and then inoculated by rubbing their adaxial (upper) surface with urediniospores on the adaxial (lower) surface of a previously infected leaf, and then leaving the infected leaf atop the inoculated leaf to form a sandwich.

The leaf sandwiches were incubated in the dark at room temperature for 2 days in plastic bags previously misted with deionized water to maintain 100% humidity. The inoculated leaves were then transferred to Magenta boxes or Mason jars containing 50 ml of water agar by inserting the petiole of each leaf into the agar to maintain a vertical position. Incubation continued for 10 days with a 12 hr photoperiod or until urediniospores were observed.

Collection of spores. By way of a detailed example, Phakopsora pachyrhizi spores were rinsed from infected leaves into 50 ml tubes containing 15 ml of sterile water and Tween 20 (0.01%) by gentle shaking. After a few minutes the spores settled to the bottom of the tube and were pipeted into microfuge tubes and concentrated by centrifugation at 10,000 rpm for 5 min. Spores were resuspended in sterile water and Tween 20 at a concentration of 10⁵/ml.

Administration of dsRNA. For each treatment 10,000 spores in 50 microliters were mixed with 50 microliters of sterile water and Tween 20 containing 30 micrograms of the different dsRNAs and incubated for 24 hours at RT.

Toxicity Assay. Following incubation with dsRNA, the spores were pipetted onto the midvein of a previously water-misted detached soybean leaf and covered with another misted leaf to form a sandwich. The sandwiches were incubated in misted plastic bags to maintain 100% humidity in the dark for 2 days at RT. The inoculated leaves were them inserted into the agar and maintained in Magenta boxes as above. Lesions were counted after pustules and urediniospores were observed. The numbers of pustules were recorded from each 3 replicates, and the percentage reduction obtained by comparison with the untreated (no-RNA) control and other dsRNAs of interest.

Treatments with siRNAs were similar except that they contained 5 μg of siRNAs in the final volume of 100 ul.

### Example 5

### Assay in Soybean Rust

Toxicity of various dsRNAs (of the indicated sequence IDs) was quantified by pustule formation by Phakopsora pachyrhizae using the Detached Soybean Leaf Assay. Replicates of 3 (averages shown below) were performed using 100 μl of solution containing 10,000 spores and 30 p1 of dsRNAs and incubated for 16 hours at 25C. The results are shown in Table 3

TABLE 3
Assay in Soybean Rust

| | RNA | # of pustules/ | % Reduction |
|---|---|---|---|
| SEQ ID | source | leaf | in pustules |
| none | No | 370 | 0 |
| | RNA | | |
| 2 | GUS | 330 | 11 |
| 8 | Gz | 320 | 14 |
| 15 | Ss | 300 | 19 |
| 12 | Fsg | 290 | 22 |
| 3 | Ps | 250 | 32 |
| 16 | Pp | 100 | 73 |

### Example 6

### Assay in Fungal Pests of Soybean

The various dsRNA molecules were tested against the soybean fungal pathogens as indicated in Table 4. The dsRNA molecules were administered to the fungi by imbibition and toxicity tested by reduction in colony number or colony growth as described above. The results are averages of 3 replicates using 50 μl of solution containing 30 μg of dsRNAs

TABLE 4
Results of RNAs on Soybean fungal pathogens.

| | | |
|---|---|---|
| Fusarium | | |
| solani | | |
| glycines | | |
| % | Sclerotinia | Phytophthora |
| reduction | sclerotium | sojae |

| | | | |
|---|---|---|---|
| SEQ | RNA | in colony | % reduction in colony |
| ID | source | formation | radial growth |

| | | | | |
|---|---|---|---|---|
| | No | 0^{x} | 0 | 0 |
| | RNA | | | |
| 2 | GUS | 12 | 8 | 7 |
| 16 | Pp | 36 | 36 | 20 |
| 8 | Gz | 61 | 40 | 13 |
| 9 | Gz | 64 | 36 | 33 |
| 12 | Fsg | 63 | 40 | 20 |
| 15 | Ss | 15 | 92 | 7 |
| 3 | Ps | 35 | 20 | 80 |

### Example 7

### siRNA Assay in Fungal Pests of Corn

Toxicity of various siRNAs, prepared as described above, was tested against Gibberella zeae and quantified by colony formation. Replicates of 3 (averages shown in Table 5) were performed with 50 μl of solution containing 5 μg of siRNAs.

TABLE 5
Toxic effects of siRNAs on colony formation by Gibberella zeae.

| | | | % Reduction |
|---|---|---|---|
| | | | in Colony |
| | | | Formation by |
| SEQ | RNA | # of | Gibberella |
| ID | source | Colonies | zeae |
| | No | 356 | 0 |
| | RNA | | |
| 2 | GUS | 298 | 16 |
| 8 | Gz | 155 | 56 |
| 10 | Gz | 126 | 64 |
| 9 | Gz | 131 | 63 |

### Example 8

### Assay in Bacterial Pests of Cabbage

RNA molecules were tested for toxic effects on the viability of Xcc cells using colony counts or using the Promega CellTiter 96 assay which measures the reduction of a tetrazolium compound by living cells.

The target genes were amplified from bacterial cultures by PCR. Oligonucleotide primers containing the T7 RNA polymerase promoter were used to synthesize the RNA molecules in vitro and incubated to form dsRNA molecules. The dsRNA molecules were cleaved using RNase III to form siRNAs.

Xcc cells (10⁶) were incubated in solutions of the dsRNA (35 μg in 30 μl) or siRNA (10 μg in 30 μl) for 16 hours and plated on YDC medium and the toxic effects were quantified.

The dsRNA and siRNAs were administered to the bacterial by incubation in the bacterial culture medium and the toxic effects were quantified.

As shown in Table 6, administration of siRNAs and dsRNAs showed substantial; toxicity to the bacteria, whether measure by colony counts (Table 6) or by the tetrazolium reduction assays (data not shown). This indicates that both dsRNAs and dsRNAs of Gene A and the 23S rRNA have high potential for conferring resistance to Xcc and Xac.

TABLE 6
the Effects of dsRNA and siRNA of Genes from Xanthomonas
campestris pv campestris (Xcc) on the Viability of Xcc.

| | # Colonies formed | % Reduction in |
|---|---|---|
| Treatment | (n = 3) | # of colonies |
| Control (no RNA) | 111 | 0 |
| Unrelated gene dsRNA | 102 | 8 |
| Unrelated gene siRNA | 100 | 10 |
| SEQ ID No. 21 dsRNA | 77 | 30 |
| SEQ ID No. 21 siRNA | 56 | 50 |
| SEQ ID No. 20 dsRNA | 84 | 24 |
| SEQ ID No. 20 siRNA | 49 | 55 |

### Example 9

### Pest Resistance in Transformed Plants

Plants are transformed with gene silencing constructs containing test sequences described above. Silencing constructs that contain sequences that demonstrated high toxicity in the assays disclosed here are found to be superior in conferring pest resistance to those that demonstrated less or no toxicity. Indeed, durability of resistance is correlated with toxicity. These results indicate the utility of these methodologies in assessing the efficacy of test sequences in gene silencing constructs to confer pest resistance in transformed plants.

We claim:

1. A method of identifying a construct useful for conferring pest resistance in a plant comprising the steps of:
(a) selecting at least one pest target nucleotide sequence;
(b) producing a plurality of test agents, wherein each test agent comprises a sense RNA molecule and an antisense RNA molecule corresponding to at least a portion of the at least one pest target nucleotide sequence;
(c) testing each of the plurality of test agents, wherein the testing of each of the plurality of test agents comprises:
i. digesting the test agent to produce smaller fragments of the test agent;
ii. administering the digested test agent to a pest; and
iii. measuring a toxic effect of the digested test agent on the pest;
wherein the measured toxic effect of a first test agent of the plurality is greater than the measured toxic effect of a second test agent of the plurality; and
(d) after the testing step, producing a silencing construct comprising an antisense sequence, wherein the silencing construct exhibits greater homology to the first test agent than to the second test agent.

2. The method of claim 1 wherein the administration step (c) further comprises addition of an RNA stabilizer.

3. The method of claim 1 wherein the administration step (c) further comprises addition of an RNA uptake enhancer.

4. The method of claim 1 wherein the pest is an insect, nematode, bacterium, fungus, or plant.

5. The method of claim 1 wherein the pest is a fungus.

6. The method of claim 1 wherein the plant is a corn plant, a soybean, a potato, a tomato, a banana, or a cotton plant.

7. The method of claim 1 wherein the plant is a corn plant and the pest is a Fusarium, a Gibberella, a Cercospora, a Puccinia, a Bipolaris, or a Cochliobolus.

8. The method of claim 1 wherein the plant is a corn plant and the pest is a Fusarium moniliforme, Gibberella zeae, a Cercospora zeae-maydis, a Puccinia sorghi, a Puccinia polysora, a Bipolaris maydis, or a Cochliobolus carbonum.

9. The method of claim 1 wherein the plant is a soy bean and the pest is a Phytophthora, a Phakopsora, a Sclerotinia, or a Fusarium.

10. The method of claim 1 wherein the plant is a soy bean plant and the pest is a Phytophthora sojae, a Phakopsora pachyrhiz, a Sclerotinia sclerotiorum, or a Fusarium solani f.sp. glycines.

11. The method of claim 1 wherein the plant is a potato plant and the pest is a Phytophthora infestans, a Alternaria solani, or a Rhizoctonia solani.

12. The method of claim 1 wherein the plant is a tomato plant and the pest is an Alternaria alternata f.sp. lycopersici, a Fusarium oxysporum f.sp. lycopersici, a Sclerotinia sclerotiorum, a Phytophthora infestans, or an Alternaria solani

13. The method of claim 1 wherein the plant is a banana plant and the pest is a Fusarium, a Mycosphaerella, or a Colletotrichum.

14. The method of claim 1 wherein the plant is a banana plant and the pest is a Fusarium oxysporum f. sp. cubense, a Mycosphaerella fijiensis, or a Colletotrichum musae.

15. The method of claim 1 wherein the plant is a cotton plant and the pest is a F. oxysporium f. sp. Vasinfectum, a Rhizoctonia solani, a Verticillium dahliae, na Ascochyta gossypi, or a Phymatotrichum omnivorum.

16. The method of claim 1 wherein the digesting of step (c) is performed using a dicer enzyme.

17. The method of claim 1 wherein the sense RNA molecule and antisense RNA molecule are joined through a phosphodiester linkage.

18. The method of claim 1 wherein the administration step (c) is accomplished by feeding, injecting, bombardment, electroporation, or incubation.

19. The method of claim 1, further comprising transforming a plant with the silencing construct.

20. The method of claim 5, further comprising transforming a plant with the silencing construct.


SEQUENCE LISTING (21 sequences)

SEQ ID NO: 1 - DNA, 685 bp, Phytophthora nicotianae
catgaaattcttcgctcgcagtagatggcactccagtcgttgccgtgtcgtcacggctcgcctacgatccgattaaccgcagacacaccgattgtgcctgattcagagaggctcccactgaaacgagatgagcctggctcacggtcgatgcgcagcaccttcataccatcttcacaatgctcgaacttgtcttcggcaacggcttcgtcatcgcgtcggcgatcatgtcgtcggtaccaacatggcgaagccgttgcctcgtcctccactaggtggcgcaccaagtggaacttgttcataatatgcttgctcttgctgtgcttgccaggcttggcagttagatacatgcacgacatgttttcgccgtgtatctccggagtcgcaaactcccagcatagttcgtcacagagtccacgtagccactgtagatctctggtaccttcattcatagcaatatactctgcttccgttgtgctctgtgcgttgatctcttgctttcttgatccgtacgaaaccacattgccattgacgaacgtgacgaactgcctaacactctttcggtcatcagggtcattgcgtagtgagcatcggtgtagcacgtcagattcacgtcgttcccggcaacaattgtccatcactagccgtggttctgcttgacactaagccgtggttctgcttgactcga

SEQ ID NO: 2 - DNA, 817 bp, Escherichia coli
gtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacctcgagaccgtggtgacgcatgtcgcgcaagactgtaaccacgcgtctgtt

SEQ ID NO: 3 - DNA, 613 bp, Phytophthora sojae
tccgtaggtgaacctgcggaaggatcattaccacgcctaaaaaactttccacgtgaaccgtatcaacaagtagttgggggcctgctctgtgtggctgtctgtcgatgtcaaagtcggcggctggctgctgtgtggcgggctctatcatggcgattggtttgggtcctcctcgtggggaactggatcatgagcccactttttaaacccattcttaaatactgaatatactgtggggacgaaagtctctgcttttaactagatagcaactttcagcagtggatgtctaggctcgcacatcgatgaagaacgctgcgaactgcgatacgtaatgcgaattgcaggattcagtgagtcatcgaaattttgaacgcatattgcacttccgggttagtcctgggagtatgcctgtatcagtgtccgtacatcaaacttggctctcttccttccgtgtagtcggtggatggagacgccagacgtgaggtgtcttgcggcgtggccttcgggctgcctgcgagtcccttgaaatgtactgaactgtacttctctttgctcgaaaagcgtgacgttgttggttgtggaggctgcctgtatggccagtcggcgaccggtttgt

SEQ ID NO: 4 - DNA, 663 bp, Puccinia sorghi
aacaaggtttctgtaggtgaacctgcagaaggatcattattaaaagaactagagtgcacttaattgtggctcgaccccttttaaactcaccccaaactttcaaagactcttttgcatggtttgtaacaaatcattgcacctgagtaaaagtaacattcttgattgaatgttacattacccacccccttttatttttccaaaactttttttttacacatacacacaagtttaaaagaatgtaaacaaccacctttaattataaaataacttttaacaatggatctctaggctctcacatcgatgaagaacacagtgaaatgtgataagtaatgtgaattgcagaattcagtgaatcatcgaatctttgaacgcatcttgcgccttttggtattccaaaaggcacacctgtttgagtgtcatgaaaccctctcacaaaataaataatttttattatgatttttgtggatgttgagtgctgctgtgttacacatagctcactttaaatgtataagtcatcttctttatatagcaaaaaagaagagatggattgacttgatgtgtaataatttttttttcatcacattgaggaaagtagcaatacttgccatctttatattattttgttgttgagatagagactactaaacaaacaatttaaaattt

SEQ ID NO: 5 - DNA, 600 bp, Phytophthora infestans
tttccgtaggtgaacctgcggaaggatcattaccacacctaaaaactttccacgtgaaccgtttcaacccaatagttgggggtcttacttggcggcggctgctggctttattgctggcggctactgctgggcgagccctatcaaaaggcgagcgtttggacttcggtctgagctagtagcttttttattttaaaccctttacttaatactgattatactgtggggacgaaagtctctgcttttaactagatagcaactttcagcagtggatgtctaggctcgcacatcgatgaagaacgctgcgaactgcgatacgtaatgcgaattgcaggattcagtgagtcatcgaaattttgaacgcatattgcacttccgggttagtcctggaagtatgcctgtatcagtgtccgtacaacaaacttggctttcttccttccgtgtagtcggtggaggagatgccagatgtgaagtgtcttgcggttggttttcggaccgactgcgagtccttttaaatgtactaaactgtacttctctttgctccaaaagtggtggcattgctggttgtggacgctgctattgtagcgagttggcgaccggttt

SEQ ID NO: 6 - DNA, 564 bp, Phytophthora infestans
gccctcgtcggctccacttccgccaccacgtgcaccacctcgcagcagaccgtagcgtacgtggcgctcgtaagcatcctctcggacacgtcgtttaatcagtgctcgacggactccggctactcgatgctgacggccacctcgctgcccacgacggagcagtacaagctcatgtgcgcgtcgacggcgtgcaagacgatgatcaacaagatcgtgtcgctcaacgctcccgactgcgagctgacggtgccaactagtggcctggtactcaacgtgttcacagccctcgtcggctccacttccgccaccacgtgcaccacctcgcagcagaccgtagcgtacgtggcgctcgtaagcatcctctcggacacgtcgtttaatcagtgctcgacggactccggctactcgatgctgacggccacctcgctgcccacgacggagcagtacaagctcatgtgcgcgtcgacggcgtgcaagacgatgatcaacaagatcgtgtcgctcaacgctcccgactgcgagctgacggtgccaactagtggcctggtactcaacgtgttcaca

SEQ ID NO: 7 - DNA, 152 bp, Gibberella zeae
caactcccaaacccctgtgaacataccttatgttgcctcggcggatcagcccgcgccccggcagtcctgctgcactccccaaatacattggcggtcacgtcgagcttccatagcgtagtaatttacacatcgttactggtaatcgtcgcggc

SEQ ID NO: 8 - DNA, 299 bp, Gibberella zeae
cgagtttacaactcccaaacccctgtgaacataccttatgttgcctcggcggatcagcccgcgccccgtaaaaagggacggcccgccgcaggaaccctaaactctgtttttagtggaacttctgagtataaaaaacaaataaatcaaaactttcaacgcccagcttggtgttgggagctgcagtcctgctgcactccccaaatacattggcggtcacgtcgagcttccatagcgtagtaatttacacatcgttactggtaatcgtcgcggccacgccgttaaaccccaacttctgaatg

SEQ ID NO: 9 - DNA, 600 bp, Gibberella zeae
tccgtaggtgaacctgcggagggatcattaccgagtttacaactcccaaacccctgtgaacataccttatgttgcctcggcggatcagcccgcgccccgtaaaaagggacggcccgccgcaggaaccctaaactctgtttttagtggaacttctgagtataaaaaacaaataaatcaaaactttcaacaacggatctcttggttctggcatcgatgaagaacgcagcaaaatgcgataagtaatgtgaattgcagaattcagtgaatcatcgaatctttgaacgcacattgcgcccgccagtattctggcgggcatgcctgttcgagcgtcatttcaaccctcaagcccagcttggtgttgggagctgcagtcctgctgcactccccaaatacattggcggtcacgtcgagcttccatagcgtagtaatttacacatcgttactggtaatcgtcgcggccacgccgttaaaccccaacttctgaatgttgacctcggatcaggtaggaatacccgctgaacttaagcatatcaataagcggaggaaaagaaaccaacagggattgccctagtaacggcgagtgaagcggcaacagctcaa

SEQ ID NO: 10 - DNA, 446 bp, Gibberella zeae
accgagtttacaactcccaaacccctgtgaacataccttatgttgcctcggcggatcagcccgcgccccgtaaaaagggacggcccgccgcaggaaccctaaactctgtttttagtggaacttctgagtataaaaaacaaataaatcaaaactttcaacaacggatctcttggttctggcatcgatgaagaacgcagcaaaatgcgataagtaatgtgaattgcagaattcagtgaatcatcgaatctttgaacgcacattgcgcccgccagtattctggcgggcatgcctgttcgagcgtcatttcaaccctcaagcccagcttggtgttgggagctgcagtcctgctgcactccccaaatacattggcggtcacgtcgagcttccatagcgtagtaatttacacatcgttactggtaatcgtcgcggccacgccgttaaacccc

SEQ ID NO: 11 - DNA, 390 bp, Gibberella moniliformis
gtgaacataccaattgttgcctcggcggatcagcccgctcccggtaaaacgggacggcccgccagaggacccctaaactctgtttctatatgtaacttctgagtaaaaactttcaacaacggatctcttggttctggcatcgatgaagaacgcagcaaaatgcgataagtaatgtgaattgcagaattcagtgaatcatcgaatctttgaacgcacattgcgcccgccagtattctggcgggcatgcctgttcgagcgtcatttcaaccctcaagcccagcttggtgttgggactcgcgagtcaaatcgcgttccccaaattgattggcggtcacgtcgagcttccatagcgtagtagtaaaaccctcgttactggtaatcgtcgcggcc

SEQ ID NO: 12 - DNA, 439 bp, Fusarium solani f. sp. Glycines
atacaactcatcaaccctgtgaacatacctaaaacgttgcttcggcgggaacagacggccctgtaacaacgggccgcccccgccagaggacccctaactctgtttttataatgtttttctgagtaaacaagcaaataaattaaaactttcaacaacggatctcttggctctggcatcgatgaagaacgcagcgaaatgcgataagtaatgtgaattgcagaattcagtgaatcatcgaatctttgaacgcacattgcgcccgccagtattctggcgggcatgcctgttcgagcgtcattacaaccctcaggcccccgggcctggcgttggggatcggcggaagccccctgtgggcacacgccgtccctcaaatacagtggcggtcccgccgcagcttccattgcgtagtagctaacacctcgcaactggagagcggcgc

SEQ ID NO: 13 - DNA, 447 bp, Cercospora zeae-maydis
ctgagtgagggccttcgggctcgacctccaaccctttgtgaacacaacttgttgcttcgggggcgaccctgccgttccgacggcgagcgcccccggaggccttcaaacactgcatctttgcgtcggagtttaagtaaattaaacaaaactttcaacaacggatctcttggttctggcatcgatgaagaacgcagcgaaatgcgataagtaatgtgaattgcagaattcagtgaatcatcgaatctttgaacgcatattgcgccctttggtattccgaagggcatgcctgttcgagcgtcatttcaccactcaagcctagcttggtattgggcgccgcggtgttccgcgcgccttaaagtctccggctgagctgtccgtctctaagcgttgtgatttcattaatcgcttcggagcgcgggcggtcgcggccgttaaatctttcacaag

SEQ ID NO: 14 - DNA, 300 bp, Glomerella graminicola
atcgagttaccgctctacaaccctttgtgaacatacctaactgttgcttcggcgggcaggggaggatacctaactctattttaacgacgtttcttctgagtggcacaagcaaataattaaaacttttaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccctcaagctctgcttggtg

SEQ ID NO: 15 - DNA, 265 bp, Sclerotinia sclerotiorum
agagttcatgcccgaaagggtagacctcccacccttgtgtattattactttgttgctttggcgagctgctcttcggggccttgtatgctcgccagagaatatcaaaactctttttattaggggggcatgcctgttcgagcgtcatttcaaccctcaagctcagcttggtattgagtccatgtcagtaatggcaggctctaaaatcagtggcggcgccgctgggtcctgaacgtagtaatatctctcgttacaggttctcggtgtg

SEQ ID NO: 16 - DNA, 558 bp, Phakopsora pachyrhizi
ataaaaagctaaagagtgcactttattgtggctcaaaactaaactttttaataaacccatttaattggctcattgattgataagatctttgggcaatggtagctttgaaaaaagctgcaacccacctattaatcataatcttttttttttttaactcaaagtcaaatagaatgttttataaatataaatatatatatataacttttaacaatggatctctaggctttcatatcgatgaagaacacagtgaaatgtgataattaatgtgaattgcagaattcagtgaatcatcaagtttttgaacgcaccttgcaccttttggtattccaaaaggtacacctgtttgagtgtcatgaaatcttctcaacattatttcttttttttaaagggaaattgttggattttgagtgttgctgttgctttttttgcagctcactttaaataaataaatatatataagtttcagtatattttgatgtaataataaaatcatttcatcaaaaaaataaatatatgtgagatttattataacattaattgaatgtaaatttttttttt

SEQ ID NO: 17 - DNA, 400 bp, Phytophthora cinnamomi
aactgttgtgcatggagcaactattgaaattatttggacttctatacctgctttaattttattaactgttgctataccatcatttgctttattatattcaatggatgaagttattgatccaattattacattaaaagtaataggtagtcaatggtattggagttatgaatattcagataatttagaattttcagatgaacctttaatttttgatagttatatggtacaagaagatgatttagctattggtcaatttagacttttagaagtagataatcgtgtagttgttccaactaatagtcatattagagttttaattacagcatcagatgttttacattcatgggctataccatcattaggtattaaattagatgcttgtcctggtcgtttaaatcaa

SEQ ID NO: 18 - DNA, 399 bp, Phytophthora cinnamomi
ccgacatcgacggcttcctcgtggtcggcgcctcgctcaagcccgacttcctgcagatcatcaacgcgcagaacccccacgccaacgtgggcggcgccgtcaacgtcgccatcaacggcttctgccgtatcggccgtctggtcctccgtgccgccgccaagaacccgctcatcaacatcgtggccatcaacgaccccttcatctccacgacctacatggagtacatgctctagtacgacacggtgcacggcaagttcgacggcacgctgtcccacgacgagcagcacatcttcgtgacggcaagcccatccgcgtcttcaacgagatgaacccggccaacatcaagtggggcgaggagcaggtgcagtacgtggtggagtccacgggcgccttcacgac

SEQ ID NO: 19 - DNA, 400 bp, Phytophthora cinnamomi
ccacgggcgagttcgaggccctggagatgcgcgatggcggcaaggcgtacatgggcaagggtgtcctgaacgccgtgaagaacgtgaacgagatcatcgcccccgctctcatcggcaaggacgtgaccaagcaggccgagcttgaccgctacatggttgagcagctcgacggcacgcagaacgagtggggctggtgcaagaagaaactgggcgccaacgccatcctgggcgtgtcgctcgcgctgtgccgcgctggtgccgccgccaagaagcagcccctgtggcagtacatcgccgacctggccggcaaccccacgccgtgcctgccggtgccgtcgttcaacatcatcaacggcggctcgcacgctgtcaacaagctggcgatgcaggagttcattat

SEQ ID NO: 20 - DNA, 400 bp, Xanthomonas campestris
atggtcaagccgcacggatcattagtatcagttagctcaatacattgctgtacttacacacctgacctatcaaccacgtagtctacatggttcctttagggggcttgtgccccgggaagtctcatcttgaggcgcgcttcccgcttagatgctttcagcggttatcgcttccgaacatagctacccggcaatgccactggcgtgacaaccggaacaccagaggttcgtccactccggtcctctcgtactaggagcagcccctctcaaacttccaacgcccatggcagatagggaccgaactgtctcacgacgttctgaacccagctcgcgtaccactttaaatggcgaacagccatacccttgggaccgactacagccccaggatgtgatgagccgacat

SEQ ID NO: 21 - DNA, 401 bp, Xanthomonas campestris
ctacgacaacggcaagtacaacctggtgggcgaaaacaagcgcctgaccagcgagcagttcgtcgacttcttggccgattgggtggcgcagtacccgatcatcagcatcgaagacggcctggccgaagacgactgggccggctggaagctgctgaccgatcgcgtcggcaagaaggtgcagctggtgggcgacgatctgttcgtcaccaacccgaagatattcaaggaaggcatcgacagcggcaccgccaacgcgatcctgatcaaggtcaaccagatcggcacgctgactgagacgctggaagccattgccatggcgcatgcggccaactacgcctcgatcgtgtcgcaccgttcgggcgagaccgaagacaccaccatcgccgatatcgccgtggcca